(19) United States
(12) Patent Application Publication
(10) Pub. No.: US 2026/0118310 A1
(43) Pub. Date: Apr. 30, 2026

(54) GAS SENSOR

(71) Applicant: NGK INSULATORS, LTD., Nagoya-shi (JP)

(72) Inventors: Atsuhiko MASUDA, Nagoya-shi (JP); Daichi ICHIKAWA, Nagoya-shi (JP)

(21) Appl. No.: 19/424,239

(22) Filed: Dec. 18, 2025

Related U.S. Application Data
(63) Continuation of application No. PCT/JP2024/017338, filed on May 10, 2024.

(30) Foreign Application Priority Data
Jul. 3, 2023 (JP) 2023-109464

(51) Int. Cl. G01N 27/41; G01N 33/00

(52) U.S. Cl. CPC G01N 27/41; G01N 33/0027

(57) ABSTRACT

A gas sensor includes a sensor element and a control device. When a predetermined condition is satisfied, the control device performs, as a refresh process, at least one of a first refresh process for controlling a main pump cell to pump in oxygen from around an outer pump electrode to around an inner pump electrode, a second refresh process for controlling a first measurement pump cell to pump in more oxygen from around the outer pump electrode to around a first measurement electrode than in first measurement pump control process, and a third refresh process for controlling a second measurement pump cell to pump in more oxygen from around the outer pump electrode to around a second measurement electrode than in second measurement pump control process.

### CROSS-REFERENCE TO RELATED APPLICATION

This application is a continuation application of PCT/JP2024/017338, filed on May 10, 2024, which claims the benefit of priority from Japanese Patent Application No. 2023-109464 filed on Jul. 3, 2023, the entire contents of which are incorporated herein by reference.

### BACKGROUND OF THE INVENTION

### 1. Field of the Invention

The present invention relates to a gas sensor.

### 2. Description of the Related Art

Conventionally, gas sensors configured to measure a water concentration and a carbon dioxide concentration in a measurement gas such as exhaust gas from an automobile are known. For example, Patent Literature 1 discloses a gas sensor including a sensor element having an oxygen-ion-conductive solid electrolyte layer and provided, inside, with a gas flow path, the gas sensor configured to measure concentrations of a water vapor component and a carbon dioxide component in the measurement gas. The gas flow path is formed such that a gas inlet, a first diffusion rate-limiting section, a buffer space, a fourth diffusion rate-limiting section, a first internal cavity, a second diffusion rate-limiting section, and a second internal cavity communicate with each other in this order. A main pump cell is configured to include a main inner pump electrode disposed in the first internal cavity and an outer pump electrode disposed on an outer surface of the sensor element. A first measurement pump cell is configured to include a first measurement inner pump electrode disposed in the second internal cavity and the outer pump electrode. A second measurement pump cell is configured to include a second measurement inner pump electrode disposed on a side opposite to the second diffusion rate-limiting section with respect to the first measurement inner pump electrode, and the outer pump electrode. In this gas sensor, an oxygen partial pressure in the first internal cavity is adjusted by the main pump cell such that water vapor and carbon dioxide components in the measurement gas are substantially completely decomposed in the first internal cavity. Then, oxygen is supplied into the second internal cavity by the first measurement pump cell such that hydrogen generated by decomposition of the water vapor component selectively burns in the second internal cavity, and a concentration of the water vapor component existing in the measurement gas is measured based on a magnitude of a current flowing at that time. In addition, oxygen is supplied near a surface of the second measurement inner pump electrode by the second measurement pump cell such that carbon monoxide generated by decomposition of the carbon dioxide component selectively burns near the surface of the second measurement inner pump electrode, and a concentration of the carbon dioxide component existing in the measurement gas is measured based on a magnitude of a current flowing at that time.

### CITATION LIST

### Patent Literature

PTL 1: Japanese patent No. 5918177

### SUMMARY OF THE INVENTION

In such gas sensors, during or after use of the gas sensor, a gas may be adsorbed onto at least one of a main inner pump electrode, a first measurement inner pump electrode, and a second measurement inner pump electrode, thereby causing a decrease in reduction capability or oxidation capability, and possibly resulting in a decrease in measurement accuracy of a water concentration and/or a carbon dioxide concentration.

A main object of the gas sensor of the present invention is to suppress a decrease in measurement accuracy of a water concentration and/or a carbon dioxide concentration in a measurement gas.

The gas sensor of the present invention has adopted the following configuration in order to achieve the above main object.

[1] The gas sensor according to the present invention is a gas sensor including a sensor element and a control device, the gas sensor configured to measure at least one of a water concentration and a carbon dioxide concentration in a measurement gas, wherein the sensor element includes: an element body having an oxygen-ion-conductive solid electrolyte layer and a measurement gas flow path provided therein, the measurement gas flow path configured to introduce and flow the measurement gas; a first pump cell including a first inner electrode disposed in a first chamber in the measurement gas flow path and a first outer electrode disposed on an outer surface of the element body; a second pump cell including a second inner electrode disposed in a second chamber located downstream of the first chamber in the measurement gas flow path and a second outer electrode disposed on the outer surface of the element body; and a third pump cell including a third inner electrode disposed in a third chamber located downstream of the second chamber in the measurement gas flow path and a third outer electrode disposed on the outer surface of the element body; wherein the control device is configured to perform: a first pump cell control process for controlling the first pump cell to pump out oxygen from around the first inner electrode to around the first outer electrode, thereby reducing water and carbon dioxide in the measurement gas in the first chamber; a second pump cell control process for controlling the second pump cell to pump in oxygen from around the second outer electrode to around the second inner electrode, thereby oxidizing hydrogen generated by reduction of water in the first chamber in the second chamber; a third pump cell control process for controlling the third pump cell to pump in oxygen from around the third outer electrode to around the third inner electrode, thereby oxidizing carbon monoxide generated by reduction of carbon dioxide in the first chamber in the third chamber; and at least one of a water concentration measurement process for measuring a water concentration in the measurement gas based on a second pump current flowing through the second pump cell in the second pump cell control process, and a carbon dioxide concentration measurement process for measuring a carbon dioxide concentration in the measurement gas based on a third pump current flowing through the third pump cell in the third pump cell control process; wherein, when a predetermined condition is satisfied, the control device is configured to perform, as a refresh process, at least one of: a first refresh process for controlling the first pump cell to pump in oxygen from around the first outer electrode to around the first inner electrode; a second refresh process for controlling the second pump cell to pump in more oxygen from around the second outer electrode to around the second inner electrode than in the second pump cell control process; and a third refresh process for controlling the third pump cell to pump in more oxygen from around the third outer electrode to around the third inner electrode than in the third pump cell control process.

In the gas sensor of the present invention, when a predetermined condition is satisfied, at least one of the first refresh process for controlling the first pump cell to pump in oxygen from around the first outer electrode to around the first inner electrode, the second refresh process for controlling the second pump cell to pump in more oxygen from around the second outer electrode to around the second inner electrode than in the second pump cell control process, and the third refresh process for controlling the third pump cell to pump in more oxygen from around the third outer electrode to around the third inner electrode than in the third pump cell control process is performed as a refresh process. By performing the first refresh process, a gas adsorbed on the first inner electrode can be oxidized and desorbed from the first inner electrode, thereby restoring a reducing capability of the first inner electrode. By performing the second refresh process, a gas adsorbed on the second inner electrode can be oxidized and desorbed from the second inner electrode, thereby restoring an oxidizing capability of the second inner electrode. By performing the third refresh process, a gas adsorbed on the third inner electrode can be oxidized and desorbed from the third inner electrode, thereby restoring an oxidizing capability of the third inner electrode. As a result, it is possible to suppress a decrease in measurement accuracy of at least one of the water concentration and the carbon dioxide concentration in the measurement gas. When the gas sensor of the present invention is mounted to an exhaust pipe of an internal combustion engine, examples of gases that may be adsorbed on at least one of the first inner electrode, the second inner electrode, and the third inner electrode include exhaust gas components contained in exhaust gas from the internal combustion engine and components derived from the exhaust gas components. Examples of the derived components include, for example, a reduced component reduced from an exhaust gas component and an oxidized component oxidized from the reduced component.

[2] In the gas sensor of the present invention (the gas sensor described in the above [1]), the control device may be configured to perform at least the first refresh process as the refresh process.

[3] In the gas sensor of the present invention (the gas sensor described in the above [1] or [2]), the predetermined condition may include a condition in which the solid electrolyte layer is activated.

[4] In the gas sensor of the present invention (the gas sensor described in any one of the above [1] to [3]), the predetermined condition may include a condition in which the first, second, and third pump cell control processes have been continuously performed for a predetermined period of time.

[5] In the gas sensor of the present invention (the gas sensor described in any one of the above [1] to [4]), at least two of the first, second, and third outer electrodes may be implemented as a single common electrode.

### BRIEF DESCRIPTION OF THE DRAWINGS

FIG. 1 is a schematic cross-sectional view schematically illustrating an example of a configuration of a gas sensor 100.

FIG. 2 is a block diagram illustrating an electrical connection relationship between a control device 95 and each cell and the like.

FIG. 3 is a flowchart illustrating an example of a processing routine.

FIG. 4 is an explanatory diagram illustrating experimental results regarding the gas sensor 100.

FIG. 5 is a flowchart illustrating an example of a processing routine of a modification.

FIG. 6 is a schematic cross-sectional view of a sensor element 201 of a modification.

### DETAILED DESCRIPTION OF THE INVENTION

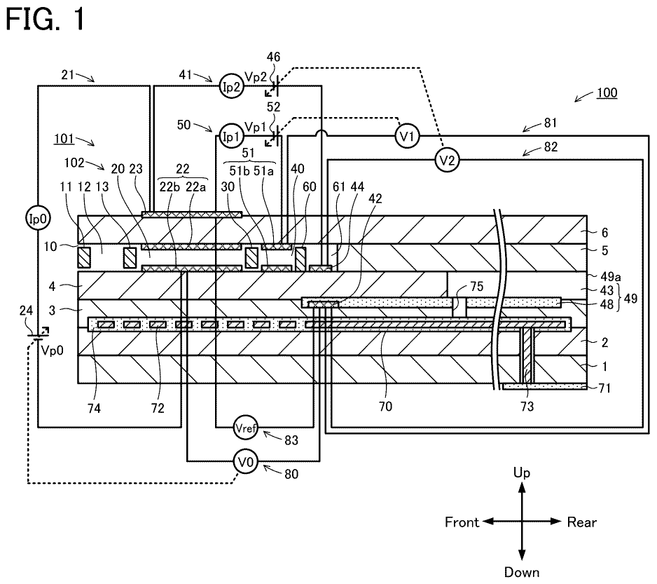

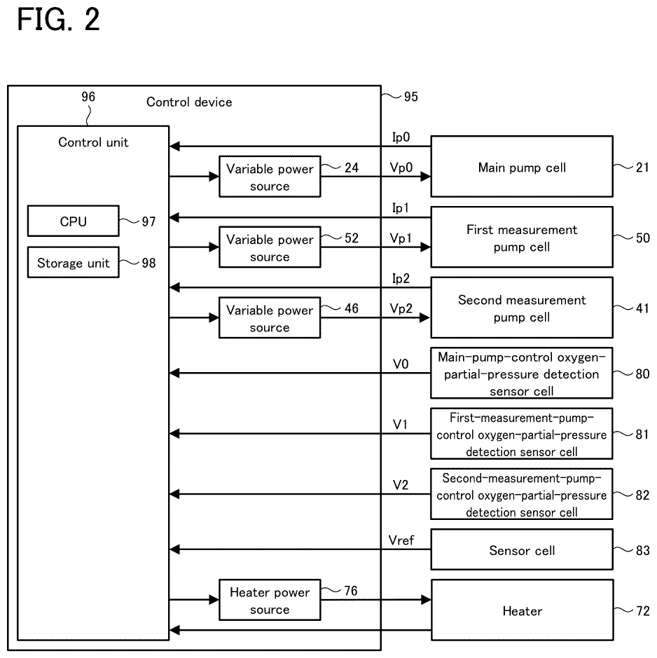

Next, embodiments of the present invention will be described with reference to the drawings. FIG. 1 is a schematic cross-sectional view schematically illustrating an example of a configuration of a gas sensor 100 according to an embodiment of the present invention. FIG. 2 is a block diagram illustrating an electrical connection relationship between a control device 95 and each cell and a heater 72. The gas sensor 100 is mounted, for example, to piping such as an exhaust pipe of an internal combustion engine. The gas sensor 100 measures a specific gas concentration, which is a concentration of a specific gas in a measurement gas, using exhaust gas from the internal combustion engine as the measurement gas. In the present embodiment, the gas sensor 100 is configured to measure a water concentration and a carbon dioxide concentration as the specific gas concentration. The gas sensor 100 includes a sensor element 101 having an element body 102 having an elongate rectangular parallelepiped shape, cells 21, 41, and 50, and cells 80 to 83 provided in the sensor element 101, a heater section 70 provided inside the sensor element 101, and a control device 95 having variable power sources 24, 46, and 52 and a heater power source 76, the control device 95 being configured to control the entire gas sensor 100. In the sensor element 101, a longitudinal direction (a left-right direction in FIG. 1) is defined as a front-rear direction, a thickness direction (an up-down direction in FIG. 1) is defined as an up-down direction, and a width direction (a direction perpendicular to the front-rear direction and the up-down direction) is defined as a left-right direction.

The element body 102 is a laminated body in which six layers, namely, a first substrate layer 1, a second substrate layer 2, a third substrate layer 3, a first solid electrolyte layer 4, a spacer layer 5, and a second solid electrolyte layer 6, each of which is made of an oxygen-ion-conductive solid electrolyte layer such as zirconia (ZrO₂), are laminated in this order from the lower side in the drawing view. The solid electrolytes forming these six layers are dense and airtight. The element body 102 is manufactured, for example, by performing predetermined processing and printing of circuit patterns on ceramic green sheets corresponding to the respective layers, laminating them, and then firing and integrating them together.

On a front end side of the sensor element 101 (the element body 102), between a lower surface of the second solid electrolyte layer 6 and an upper surface of the first solid electrolyte layer 4, a gas inlet 10, a first diffusion rate-limiting section 11, a buffer space 12, a second diffusion rate-limiting section 13, a first internal cavity 20, a third diffusion rate-limiting section 30, a second internal cavity 40, a fourth diffusion rate-limiting section 60, and a third internal cavity 61 are adjacently formed to communicate with one another in this order.

The gas inlet 10, the buffer space 12, the first internal cavity 20, the second internal cavity 40, and the third internal cavity 61 are spaces inside the sensor element 101, which are partitioned at an upper side by the lower surface of the second solid electrolyte layer 6, at a lower side by the upper surface of the first solid electrolyte layer 4, and at side portions by side surfaces of the spacer layer 5, in a manner in which the spacer layer 5 is hollowed out.

Each of the first diffusion rate-limiting section 11, the second diffusion rate-limiting section 13, and the third diffusion rate-limiting section 30 is provided as two horizontally elongated slits (with openings oriented along the longitudinal direction perpendicular to the drawing plane). The fourth diffusion rate-limiting section 60 is provided as a single horizontally elongated slit (with openings oriented along the longitudinal direction perpendicular to the drawing plane) formed as a gap between the lower surface of the second solid electrolyte layer 6 and the spacer layer 5. A portion extending from the gas inlet 10 to the third internal cavity 61 is also referred to as a measurement gas flow path.

The sensor element 101 (element body 102) includes a reference gas introduction portion 49 configured to introduce a reference gas from outside the sensor element 101 to a reference electrode 42 when measuring a specific gas concentration. The reference gas introduction portion 49 includes a reference gas introduction space 43 and a reference gas introduction layer 48. The reference gas introduction space 43 is a space provided inwardly from a rear end surface of the sensor element 101. The reference gas introduction space 43 is located between an upper surface of a third substrate layer 3 and a lower surface of a spacer layer 5, and is laterally defined by side surfaces of a first solid electrolyte layer 4. The reference gas introduction space 43 opens to the rear end surface of the sensor element 101, and this opening functions as an inlet portion 49a of the reference gas introduction portion 49. The reference gas is introduced into the reference gas introduction space 43 through the inlet portion 49a. The reference gas introduction portion 49 introduces the reference gas, which has been introduced through the inlet portion 49a, to the reference electrode 42 while imparting a predetermined diffusion resistance. In the present embodiment, the reference gas is ambient air.

The reference gas introduction layer 48 is provided between the upper surface of the third substrate layer 3 and the lower surface of the first solid electrolyte layer 4. The reference gas introduction layer 48 is a porous body made of a ceramic material such as alumina. A portion of the upper surface of the reference gas introduction layer 48 is exposed within the reference gas introduction space 43. The reference gas introduction layer 48 is formed to cover the reference electrode 42. The reference gas introduction layer 48 allows the reference gas to flow from the reference gas introduction space 43 to the reference electrode 42.

The reference electrode 42 is an electrode formed between the upper surface of the third substrate layer 3 and the first solid electrolyte layer 4. As described above, the reference gas introduction layer 48 connected to the reference gas introduction space 43 is provided around the reference electrode 42. Further, as will be described later, the reference electrode 42 enables measurement of oxygen concentration (oxygen partial pressure) within the first internal cavity 20, the second internal cavity 40, and the third internal cavity 61. The reference electrode 42 is formed as a porous cermet electrode (for example, a cermet electrode composed of Pt and ZrO2).

In the measurement gas flow path, the gas inlet 10 is a portion that opens to the external space, allowing the measurement gas to be introduced into the sensor element 101 from the external space through the gas inlet 10. The first diffusion rate-limiting section 11 is a portion that imparts a predetermined diffusion resistance to the measurement gas introduced through the gas inlet 10. The buffer space 12 is a space provided to guide the measurement gas introduced through the first diffusion rate-limiting section 11 to the second diffusion rate-limiting section 13. The second diffusion rate-limiting section 13 is a portion that imparts a predetermined diffusion resistance to the measurement gas introduced from the buffer space 12 into the first internal cavity 20. When the measurement gas is introduced from the outside of the sensor element 101 into the first internal cavity 20, the measurement gas rapidly drawn into the sensor element 101 through the gas inlet 10 due to pressure fluctuations in the external space (for example, exhaust pressure pulsations when the measurement gas is exhaust gas from an automobile) is not directly introduced into the first internal cavity 20 but is introduced into the first internal cavity 20 after the pressure fluctuations of the measurement gas are attenuated through the first diffusion rate-limiting section 11, the buffer space 12, and the second diffusion rate-limiting section 13. As a result, the pressure fluctuations of the measurement gas introduced into the first internal cavity 20 become almost negligible. The first internal cavity 20 is provided as a space for adjusting the oxygen partial pressure in the measurement gas introduced through the second diffusion rate-limiting section 13. This oxygen partial pressure is adjusted by the operation of the main pump cell 21.

The main pump cell 21 is an electrochemical pump cell comprising an inner pump electrode 22 having a ceiling electrode portion 22a provided substantially over the entire lower surface of the second solid electrolyte layer 6 facing the first internal cavity 20, an outer pump electrode 23 provided on a region corresponding to the ceiling electrode portion 22a on the upper surface of the second solid electrolyte layer 6 in a manner that exposed outside the sensor element, and the second solid electrolyte layer 6, the spacer layer 5, and the first solid electrolyte layer 4 which form a current path between these electrodes.

The inner pump electrode 22 is formed across the upper and lower solid electrolyte layers (the second solid electrolyte layer 6 and the first solid electrolyte layer 4) that define the first internal cavity 20 and across the spacer layer 5 that provides the sidewalls. Specifically, a ceiling electrode portion 22a is formed on the lower surface of the second solid electrolyte layer 6 that defines the ceiling surface of the first internal cavity 20, and a bottom electrode portion 22b is formed on the upper surface of the first solid electrolyte layer 4 that defines the bottom surface. In addition, a side electrode portion (not shown) connecting the ceiling electrode portion 22a and the bottom electrode portion 22b is formed on the inner side surfaces of the spacer layer 5 that form the side walls of the first internal cavity 20. The electrode structure thus forms a tunnel-like configuration at the locations where the side electrode portions are disposed.

In the main pump cell 21, by applying a desired voltage Vp0 between the inner pump electrode 22 and the outer pump electrode 23, and flowing a pump current Ip0 in a positive or negative direction between the inner pump electrode 22 and outer pump electrode 23, it is possible to pump out oxygen in the first internal cavity 20 to the external space or to pump in oxygen from the external space into the first internal cavity 20.

Further, in order to detect the oxygen concentration (oxygen partial pressure) in the atmosphere within the first internal cavity 20, an electrochemical sensor cell, namely, a main-pump-control oxygen-partial-pressure detection sensor cell 80 is configured by the inner pump electrode 22, the second solid electrolyte layer 6, the spacer layer 5, the first solid electrolyte layer 4, the third substrate layer 3, and the reference electrode 42.

By measuring an electromotive force (voltage V0) in the main-pump-control oxygen-partial-pressure detection sensor cell 80, the oxygen concentration (oxygen partial pressure) in the first internal cavity 20 can be determined. Furthermore, the pump current Ip0 is controlled by feedback-controlling the voltage Vp0 of the variable power source 24 such that the voltage V0 becomes a target value. As a result, the oxygen concentration in the first internal cavity 20 is adjusted.

The third diffusion rate-limiting section 30 is a portion that imparts a predetermined diffusion resistance to the measurement gas in which the oxygen concentration (oxygen partial pressure) has been controlled by operation of the main pump cell 21 in the first internal cavity 20, and guides the measurement gas to the second internal cavity 40.

The second internal cavity 40 is provided as a space in which the oxygen partial pressure of the measurement gas introduced through the third diffusion rate-limiting section 30 is adjusted by the first measurement pump cell 50 and process related to measurement of a water concentration in the measurement gas is performed.

The first measurement pump cell 50 is an electrochemical pump cell constituted by a first measurement electrode 51 having a ceiling electrode portion 51a provided over substantially the entire lower surface of the second solid electrolyte layer 6 facing the second internal cavity 40, an outer pump electrode 23 (not limited to the outer pump electrode 23 and any suitable electrode provided on an outer surface of the sensor element 101 suffices), the second solid electrolyte layer 6, the spacer layer 5, and the first solid electrolyte layer 4.

The first measurement electrode 51 is disposed in the second internal cavity 40 in a tunnel-like configuration similar to that of the inner pump electrode 22 provided in the first internal cavity 20 described above. That is, the ceiling electrode portion 51a is formed on the second solid electrolyte layer 6 that provides a ceiling surface of the second internal cavity 40, and a bottom electrode portion 51b is formed on the first solid electrolyte layer 4 that provides a bottom surface of the second internal cavity 40, and a side electrode portion (not shown) that connects the ceiling electrode portion 51a and the bottom electrode portion 51b is formed on both wall surfaces of the spacer layer 5 that provide side walls of the second internal cavity 40, the structure being in a tunnel-like configuration.

In the first measurement pump cell 50, by applying a desired voltage Vp1 between the first measurement electrode 51 and the outer pump electrode 23, it becomes possible to pump out oxygen in an atmosphere in the second internal cavity 40 to an external space, or to pump in oxygen from the external space into the second internal cavity 40.

Further, in order to control the oxygen partial pressure in an atmosphere within the second internal cavity 40, an electrochemical sensor cell, namely, a first-measurement-pump-control oxygen-partial-pressure detection sensor cell 81, is configured by the first measurement electrode 51, the reference electrode 42, the second solid electrolyte layer 6, the spacer layer 5, the first solid electrolyte layer 4, and the third substrate layer 3.

In addition, the first measurement pump cell 50 performs pumping by the variable power source 52 whose voltage is controlled based on the electromotive force (voltage V1) detected by the first-measurement-pump-control oxygen-partial-pressure detection sensor cell 81. As a result, the oxygen partial pressure in an atmosphere within the second internal cavity 40 is adjusted by a pump current Ip1 flowing through the first measurement pump cell 50.

The fourth diffusion rate-limiting section 60 is a portion that imparts a predetermined diffusion resistance to the measurement gas in which the oxygen concentration (oxygen partial pressure) has been controlled by operation of the first measurement pump cell 50 in the second internal cavity 40, and guides the measurement gas to the third internal cavity 61.

The third internal cavity 61 is provided as a space in which the oxygen partial pressure of the measurement gas introduced through the fourth diffusion rate-limiting section 60 is adjusted by the second measurement pump cell 41 and process related to measurement of a carbon dioxide concentration in the measurement gas is performed.

The second measurement pump cell 41 is an electrochemical pump cell constituted by a second measurement electrode 44 provided on an upper surface of the first solid electrolyte layer 4 facing the third internal cavity 61, the outer pump electrode 23 (not limited to the outer pump electrode 23 and any suitable electrode provided on an outer surface of the sensor element 101 suffices), the second solid electrolyte layer 6, the spacer layer 5, and the first solid electrolyte layer 4.

In the second measurement pump cell 41, by applying a desired voltage Vp2 between the second measurement electrode 44 and the outer pump electrode 23, it becomes possible to pump out oxygen in an atmosphere within the third internal cavity 61 to an external space, or to pump in oxygen from the external space into the second internal cavity 40.

Further, in order to detect the oxygen partial pressure around the second measurement electrode 44, an electrochemical sensor cell, namely, a second-measurement-pump-control oxygen-partial-pressure detection sensor cell 82, is configured by the first solid electrolyte layer 4, the third substrate layer 3, the second measurement electrode 44, and the reference electrode 42.

In addition, the variable power source 46 is controlled based on the electromotive force (voltage V2) detected by the second-measurement-pump-control oxygen-partial-pressure detection sensor cell 82, and the voltage Vp2 of the variable power source 46 is applied to the second measurement pump cell 41. As a result, the oxygen partial pressure in an atmosphere within the third internal cavity 61 is adjusted by a pump current Ip2 flowing through the second measurement pump cell 41.

Furthermore, an electrochemical sensor cell 83 is configured by the second solid electrolyte layer 6, the spacer layer 5, the first solid electrolyte layer 4, the third substrate layer 3, the outer pump electrode 23, and the reference electrode 42, and it is possible to detect the oxygen partial pressure in the measurement gas outside the sensor based on an electromotive force (voltage Vref) obtained by the sensor cell 83.

Here, each of the electrodes 22, 23, 42, 44, and 51 will be described. The inner pump electrode 22, the first measurement electrode 51, and the second measurement electrode 44 each contains a first kind of noble metal having catalytic activity. As the first kind of noble metal, at least one of Pt, Rh, Ir, Ru, and Pd can be exemplified. The outer pump electrode 23 and the reference electrode 42 also contain the first kind of noble metal. It is preferable that the first measurement electrode 51 further contains a second kind of noble metal that suppresses the catalytic activity of the first kind of noble metal with respect to carbon monoxide. By containing the second kind of noble metal, the first measurement electrode 51 has a weakened oxidizing capability with respect to carbon monoxide. As the second kind of noble metal, for example, Au can be exemplified. The inner pump electrode 22 and the second measurement electrode 44 do not contain the second kind of noble metal. It is also preferable that the outer pump electrode 23 and the reference electrode 42 do not contain the second kind of noble metal. Each of the electrodes 22, 23, 42, 44, and 51 is preferably a cermet containing a noble metal and an oxide having oxygen-ion conductivity (for example, ZrO2). Each of the electrodes 22, 23, 42, 44, and 51 is preferably porous. In the present embodiment, the first measurement electrode 51 is a porous cermet electrode composed of Pt and ZrO₂ and containing 1% Au. The inner pump electrode 22, the outer pump electrode 23, the reference electrode 42, and the second measurement electrode 44 are each porous cermet electrodes composed of Pt and ZrO₂.

The sensor element 101 includes a heater section 70 having a temperature control function that heats and maintains temperature the sensor element 101 to enhance the oxygen-ion conductivity of the solid electrolyte. The heater section 70 includes a heater connector electrode 71, a heater 72, a through-hole 73, a heater insulating layer 74, and a pressure release hole 75.

The heater connector electrode 71 is an electrode formed in a manner to be in contact with a lower surface of the first substrate layer 1. By connecting the heater connector electrode 71 to a heater power source 76 (see FIG. 2), electric power can be supplied from the heater power source 76 to the heater section 70.

The heater 72 is an electrical resistor formed in a manner to be sandwiched from above and below by the second substrate layer 2 and the third substrate layer 3. The heater 72 is connected to the heater connector electrode 71 through the through-hole 73, and generates heat when supplied with power from the heater power source 76 through the heater connector electrode 71, thereby heating and maintaining temperature the solid electrolyte forming the sensor element 101.

The heater 72 is embedded over the entire region from the first internal cavity 20 to the third internal cavity 61, and thus the entire sensor element 101 can be adjusted to a temperature at which the solid electrolyte described above becomes activated.

The heater insulating layer 74 is an insulating layer formed on upper and lower surfaces of the heater 72 by an insulating material such as alumina. The heater insulating layer 74 is provided for the purpose of obtaining electrical insulation between the second substrate layer 2 and the heater 72, and between the third substrate layer 3 and the heater 72.

The pressure release hole 75 is a portion formed to penetrate the third substrate layer 3 and the reference gas introduction layer 48 and to communicate with the reference gas introduction space 43, and is provided for the purpose of relieving an internal pressure increase associated with temperature rise within the heater insulating layer 74.

As shown in FIG. 2, the control device 95 includes the above-described variable power sources 24, 46, and 52, the above-described heater power source 76, and a control unit 96. The control unit 96 is a microprocessor including a CPU 97 and a storage unit 98. The storage unit 98 is a nonvolatile memory whose information can be rewritten and can store, for example, various programs and various data. The control unit 96 receives as inputs a voltage V0 of the main-pump-control oxygen-partial-pressure detection sensor cell 80, a voltage V1 of the first-measurement-pump-control oxygen-partial-pressure detection sensor cell 81, a voltage V2 of the second-measurement-pump-control oxygen-partial-pressure detection sensor cell 82, a voltage Vref of the sensor cell 83, a pump current Ip0 flowing through the main pump cell 21, a pump current Ip1 flowing through the first measurement pump cell 50, and a pump current Ip2 flowing through the second measurement pump cell 41. The control unit 96 controls voltages Vp0, Vp1, and Vp2 output from the variable power sources 24, 52, and 46 by outputting control signals to the variable power sources 24, 52, and 46, thereby controlling the main pump cell 21, the first measurement pump cell 50, and the second measurement pump cell 41. The control unit 96 controls power supplied from the heater power source 76 to the heater 72 by outputting a control signal to the heater power source 76. Target values V0*, V1*, and V2* described later are also stored in the storage unit 98. The CPU 97 of the control unit 96 performs control of the cells 21, 50, and 41 with reference to the target values V0*, V1*, and V2*.

The control unit 96 performs a main pump control process (an example of the first pump cell control process) for controlling the main pump cell 21 to pump out oxygen from around the inner pump electrode 22 to around the outer pump electrode 23. Specifically, the control unit 96 controls the main pump cell 21 by feedback-controlling the voltage Vp0 of the variable power source 24 such that the voltage V0 becomes a target value V0*. The target value V0* is defined as a value such that the oxygen concentration in the first internal cavity 20 becomes a predetermined low concentration sufficiently low to substantially entirely reduce water and carbon dioxide in the measurement gas. By performing this main pump control process, in the first internal cavity 20, water in the measurement gas is reduced to generate hydrogen and oxygen, and carbon dioxide in the measurement gas is reduced to generate carbon monoxide and oxygen. The generated oxygen is pumped out from around the inner pump electrode 22 to around the outer pump electrode 23 by the pump current Ip0 flowing through the main pump cell 21.

The control unit 96 performs a first measurement pump control process (an example of the second pump cell control process) for controlling the first measurement pump cell 50 to pump in oxygen from around the outer pump electrode 23 to around the first measurement electrode 51.

Specifically, the control unit 96 controls the first measurement pump cell 50 by feedback-controlling the voltage Vp1 of the variable power source 52 such that the voltage V1 becomes a target value V1*. The target value V1* is defined as a value such that the oxygen concentration in the second internal cavity 40 becomes a predetermined concentration sufficient to substantially entirely oxidize hydrogen in the second internal cavity 40. By performing this first measurement pump control process, in the second internal cavity 40, hydrogen produced by reduction of water in the first internal cavity 20 is oxidized and water is generated again. At this time, a pump current Ip1 flowing through the first measurement pump cell 50 correlates with an amount of oxygen pumped into the second internal cavity 40 to oxidize the hydrogen in the second internal cavity 40, and hence correlates with an amount of water in the measurement gas in the first internal cavity 20, which is a source for generation of the hydrogen in the second internal cavity 40. Accordingly, the pump current Ip1 correlates with a water concentration in the measurement gas, and a water concentration in the measurement gas can be measured based on the pump current Ip1. Hereinafter, a process of measuring the water concentration in the measurement gas based on such a pump current Ip1 is referred to as a water concentration measurement process.

The control unit 96 performs a second measurement pump control process (an example of the third pump cell control process) for controlling the second measurement pump cell 41 to pump in oxygen from around the outer pump electrode 23 to around the second measurement electrode 44. Specifically, the control unit 96 controls the second measurement pump cell 41 by feedback-controlling the voltage Vp2 of the variable power source 46 such that the voltage V2 becomes a target value V2*. The target value V2* is defined as a value such that the oxygen concentration in the third internal cavity 61 becomes a predetermined concentration sufficient to substantially entirely oxidize carbon monoxide in the third internal cavity 61. By performing this second measurement pump control process, in the third internal cavity 61, carbon monoxide produced by reduction of carbon dioxide in the first internal cavity 20 is oxidized and carbon dioxide is generated again. At this time, a pump current Ip2 flowing through the second measurement pump cell 41 correlates with an amount of oxygen pumped into the third internal cavity 61 to oxidize the carbon monoxide in the third internal cavity 61, and hence correlates with an amount of carbon dioxide in the measurement gas in the first internal cavity 20, which is a source for generation of the carbon monoxide in the third internal cavity 61. Accordingly, the pump current Ip2 correlates with a carbon dioxide concentration in the measurement gas, and a carbon dioxide concentration in the measurement gas can be measured based on the pump current Ip2. Hereinafter, a process of measuring the carbon dioxide concentration in the measurement gas based on such a pump current Ip2 is referred to as a carbon dioxide concentration measurement process.

Incidentally, in the second internal cavity 40, both hydrogen and carbon monoxide generated in the first internal cavity 20 arrive. However, between hydrogen and carbon monoxide, hydrogen has a faster gas diffusion rate and also more readily combines with oxygen. Therefore, in the second internal cavity 40, hydrogen among hydrogen and carbon monoxide can be selectively oxidized by the first measurement pump control process. Then, since hydrogen hardly reaches the third internal cavity 61 downstream of the second internal cavity 40, carbon monoxide can be oxidized in the second measurement pump control process. Further, in the present embodiment, as described above, the first measurement electrode 51 contains the second kind of noble metal, such that an oxidizing capability with respect to carbon monoxide is weakened. Therefore, around the first measurement electrode 51, that is, in the second internal cavity 40, hydrogen among hydrogen and carbon monoxide can be oxidized more selectively by the first measurement pump control process.

The control unit 96 performs a heater control process of outputting a control signal to the heater power source 76 to control the temperature of the heater 72 to reach a target temperature (for example, 800° C.). Here, the target temperature of the heater 72 is defined as a temperature obtained by adding a margin to a temperature at which the solid electrolyte described above is activated. The temperature of the heater 72 can be expressed by a linear function of a resistance value of the heater 72. Therefore, in the heater control process, the control unit 96 calculates a resistance value of the heater 72 as a value that can be regarded as the temperature of the heater 72 (a value convertible into the temperature), and feedback-controls the heater power source 76 such that the calculated resistance value becomes a target resistance value (a resistance value corresponding to the target temperature). The control unit 96 can obtain a voltage of the heater 72 and a current flowing through the heater 72, and calculate the resistance value of the heater 72 based on the obtained voltage and current. The control unit 96 may calculate the resistance value of the heater 72, for example, by a three-terminal method or a four-terminal method. When supplying electric power to the heater 72, the heater power source 76 adjusts power supplied to the heater 72, for example, by changing a voltage value applied to the heater 72 based on a control signal from the control unit 96.

Incidentally, including the variable power sources 24, 46, and 52 and the heater power source 76 shown in FIG. 2, the control device 95 is actually connected to each electrode inside the sensor element 101 through lead wires (not shown) formed inside the sensor element 101 and connector electrodes (not shown) formed on a rear end side of the sensor element 101 (only the heater connector electrode 71 is shown in FIG. 1).

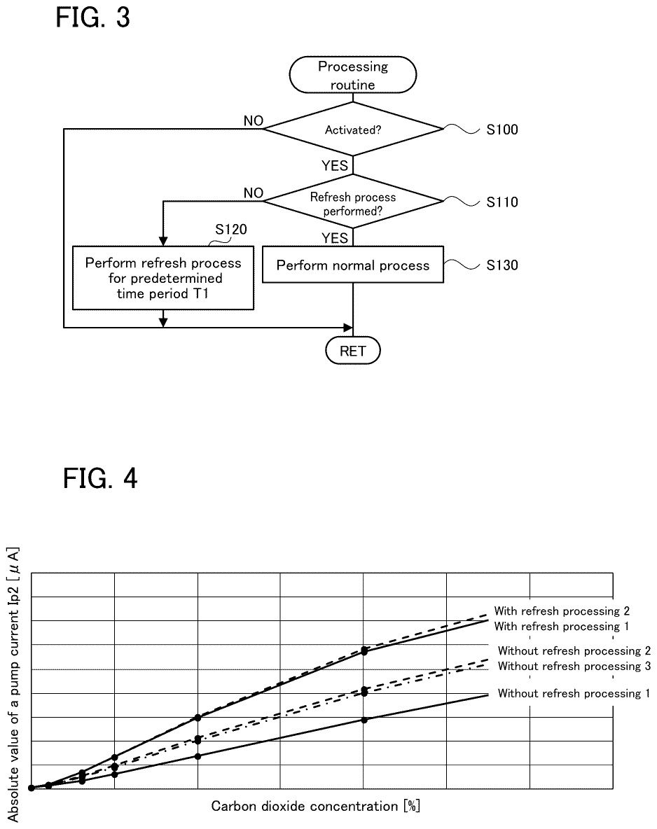

Next, an example of processing of the control unit 96 of the gas sensor 100 will be described. FIG. 3 is a flowchart illustrating an example of a processing routine executed by the CPU 97 of the control unit 96. This routine is stored, for example, in the storage unit 98 of the control unit 96 and is repeatedly executed by the CPU 97. When the gas sensor 100 is used, the CPU 97 controls the temperature of the heater 72 to reach a target temperature (for example, 800° C.) by the heater control process. In the present embodiment, a period from the start to the end of the heater control process is defined as one use of the gas sensor 100.

When the processing routine shown in FIG. 3 is executed, the CPU 97 first determines whether or not a solid electrolyte of the sensor element 101 is activated (step S100). This processing can be performed, for example, by determining whether or not a resistance value of the heater 72 is equal to or less than a predetermined resistance value. The predetermined resistance value is defined in advance as a resistance value corresponding to a temperature at which the solid electrolyte is activated (a value higher than the above-described target resistance value). When the CPU 97 determines that the solid electrolyte of the sensor element 101 is not activated, the present routine is terminated.

When the CPU 97 determines in step S100 that the solid electrolyte of the sensor element 101 is activated, the CPU 97 determines whether or not a refresh process has been performed in the present use of the gas sensor 100 (step S110). When the CPU 97 determines that the refresh process has not been performed in the present use of the gas sensor 100, the CPU 97 performs a refresh process for a predetermined time period T1 (step S120) and then terminates the present routine.

Here, as the predetermined time period T1, for example, several seconds to several minutes is used. In the refresh process, the CPU 97 performs a first refresh process, a second refresh process, and a third refresh process. The first refresh process is a process for controlling the main pump cell 21 to pump in oxygen from around the outer pump electrode 23 to around the inner pump electrode 22. The second refresh process is a process for controlling the first measurement pump cell 50 to pump in more oxygen from around the outer pump electrode 23 to around the first measurement electrode 51 than in the first measurement pump control process. The third refresh process is a process for controlling the second measurement pump cell 41 to pump in more oxygen from around the outer pump electrode 23 to around the second measurement electrode 44 than in the second measurement pump control process.

Specifically, in the first refresh process, the CPU 97 controls the main pump cell 21 by feedback-controlling a voltage Vp0 of the variable power source 24 such that a voltage V0 becomes a target value V0r* whose absolute value is smaller than a target value V0*. The target value V0r* is defined as a value such that an oxygen concentration in the first internal cavity 20 becomes higher than that during performance of the main pump control process. During performance of the first refresh process, a direction of a pump current Ip0 is opposite to that during performance of the main pump control process. In the second refresh process, the control unit 96 controls the first measurement pump cell 50 by feedback-controlling a voltage Vp1 of the variable power source 52 such that a voltage V1 becomes a target value V1r* whose absolute value is smaller than a target value V1*. The target value V1r* is defined as a value such that an oxygen concentration in the second internal cavity 40 becomes higher (closer to the reference gas) than that during performance of the first measurement pump control process. In the third refresh process, the control unit 96 controls the second measurement pump cell 41 by feedback-controlling a voltage Vp2 of the variable power source 46 such that a voltage V2 becomes a target value V2r* whose absolute value is smaller than a target value V2*. The target value V2r* is defined as a value such that an oxygen concentration in the third internal cavity 61 becomes higher (closer to the reference gas) than that during performance of the second measurement pump control process.

In the gas sensor 100, during or after use of the gas sensor 100, at least one of the inner pump electrode 22, the first measurement electrode 51, and the second measurement electrode 44 may adsorb gas, thereby causing a decrease in reducing capability or oxidizing capability and possibly resulting in a decrease in measurement accuracy of water concentration and carbon dioxide concentration. In contrast, in the present embodiment, by performing the first refresh process, a gas adsorbed on the inner pump electrode 22 can be oxidized and desorbed from the inner pump electrode 22, thereby restoring a reducing capability of the inner pump electrode 22. By performing the second refresh process, a gas adsorbed on the first measurement electrode 51 can be oxidized and desorbed from the first measurement electrode 51, thereby restoring an oxidizing capability of the first measurement electrode 51. By performing the third refresh process, a gas adsorbed on the second measurement electrode 44 can be oxidized and desorbed from the second measurement electrode 44, thereby restoring an oxidizing capability of the second measurement electrode 44. As a result, it is possible to suppress a decrease in measurement accuracy of water concentration and carbon dioxide concentration in the measurement gas. In the present embodiment, it is assumed that the gas sensor 100 is mounted to an exhaust pipe of an internal combustion engine. In this case, examples of gases that may be adsorbed on at least one of the inner pump electrode 22, the first measurement electrode 51, and the second measurement electrode 44 include exhaust gas components contained in exhaust gas from the internal combustion engine and components derived from the exhaust gas components. Examples of the derived components include, for example, a reduced component reduced from an exhaust gas component and an oxidized component oxidized from the reduced component.

When the CPU 97 determines in step S110 that the refresh process has already been performed in the present use of the gas sensor 100, the CPU 97 performs a normal process (step S130) and terminates the present routine. Here, in the normal process, the CPU 97 performs the main pump control process, the first measurement pump control process, the second measurement pump control process, the water concentration measurement process, and the carbon dioxide concentration measurement process.

FIG. 4 is an explanatory diagram illustrating experimental results of the gas sensor 100. In the figure, the horizontal axis indicates a carbon dioxide concentration, and the vertical axis indicates an absolute value of a pump current Ip2. As an experiment of the gas sensor 100, the inventors sequentially performed a preparation process, a first experimental process, a preparation process, a second experimental process, a preparation process, a third experimental process, a preparation process, a fourth experimental process, a preparation process, and a fifth experimental process. In each of the five preparation processes, the gas sensor 100 including the sensor element 101 was mounted to an exhaust pipe of an internal combustion engine such that a tip-side portion of the sensor element 101 protruded into the exhaust pipe, and the internal combustion engine was operated for several hours to several tens of hours while performing, for the gas sensor 100, a heater control process, a main pump control process, a first measurement pump control process, a second measurement pump control process, a water concentration measurement process, and a carbon dioxide concentration measurement process. After the operation of the internal combustion engine was completed, the gas sensor 100 was detached. In the first, second, and fourth experimental processes (indicated as “without refresh process 1, 2, and 3” in FIG. 4), the gas sensor 100 was mounted to a pipe such that a tip-side portion of the sensor element 101 protruded into the inside of the pipe, the solid electrolyte of the sensor element 101 was activated by the heater control process, and without performing the refresh process, the carbon dioxide concentration was gradually increased and the pump current Ip2 was detected at each concentration (step S130). Subsequently, the heater control process was terminated, and the gas sensor 100 was detached from the pipe. In the third and fifth experimental processes (indicated as “with refresh process 1 and 2” in FIG. 4), the gas sensor 100 was mounted to a pipe such that a tip-side portion of the sensor element 101 protruded into the inside of the pipe, the solid electrolyte of the sensor element 101 was activated by the heater control process, and after performing the refresh process (step S120), the carbon dioxide concentration was gradually increased and the pump current Ip2 was detected at each concentration (step S130). Subsequently, the heater control process was terminated, and the gas sensor 100 was detached from the pipe. In the first to fifth experimental processes, as a model gas, a gas in which nitrogen was used as a base gas and the carbon dioxide concentration was gradually changed was used.

From FIG. 4, it was found that, in the third and fifth experimental processes, variations among the experimental processes in a relationship between the carbon dioxide concentration and the absolute value of the pump current Ip2 were small. On the other hand, in the first, second, and fourth experimental processes, compared with the third and fifth experimental processes, variations among the experimental processes in the relationship between the carbon dioxide concentration and the absolute value of the pump current Ip2 were large, and the absolute value of the pump current Ip2 at the same carbon dioxide concentration became smaller. From these facts, it is assumed that, in the first, second, and fourth experimental processes, a decrease in measurement accuracy of a carbon dioxide concentration in the measurement gas is expected as compared with the third and fifth experimental processes. In other words, in the third and fifth experimental processes, by performing the refresh process, it is possible to suppress a decrease in measurement accuracy of a carbon dioxide concentration in the measurement gas as compared with the first, second, and fourth experimental processes. Similarly, when the refresh process is performed, it is considered that a decrease in measurement accuracy of a water concentration in the measurement gas can also be suppressed as compared with a case where the refresh process is not performed.

Here, a correspondence relationship between the components of the present embodiment and the components of the present invention will be clarified. The sensor element 101 of the present embodiment corresponds to the sensor element of the present invention, and the control device 95 corresponds to the control device. The element body 102 corresponds to the element body, the first internal cavity 20 corresponds to the first chamber, the inner pump electrode 22 corresponds to the first inner electrode, and the main pump cell 21 corresponds to the first pump cell. The second internal cavity 40 corresponds to the second chamber, the first measurement electrode 51 corresponds to the second inner electrode, and the first measurement pump cell 50 corresponds to the second pump cell. The third internal cavity 61 corresponds to the third chamber, the second measurement electrode 44 corresponds to the third inner electrode, and the second measurement pump cell 41 corresponds to the third pump cell. The outer pump electrode 23 corresponds to the first outer electrode, the second outer electrode, and the third outer electrode. The main pump control process corresponds to the first pump cell control process, the first measurement pump control process corresponds to the second pump cell control process, and the second measurement pump control process corresponds to the third pump cell control process.

According to the gas sensor 100 of the present embodiment described in detail above, when the solid electrolyte of the sensor element 101 is activated, the control device 95 performs a refresh process, specifically, the first to third refresh process. As a result, it is possible to restore the reducing capability and oxidizing capability of the inner pump electrode 22, the first measurement electrode 51, and the second measurement electrode 44. Accordingly, it is possible to suppress a decrease in measurement accuracy of water concentration and carbon dioxide concentration in the measurement gas.

It should be understood that the present invention is not limited to the embodiment described above, and various modifications may be made as long as they fall within the technical scope of the present invention.

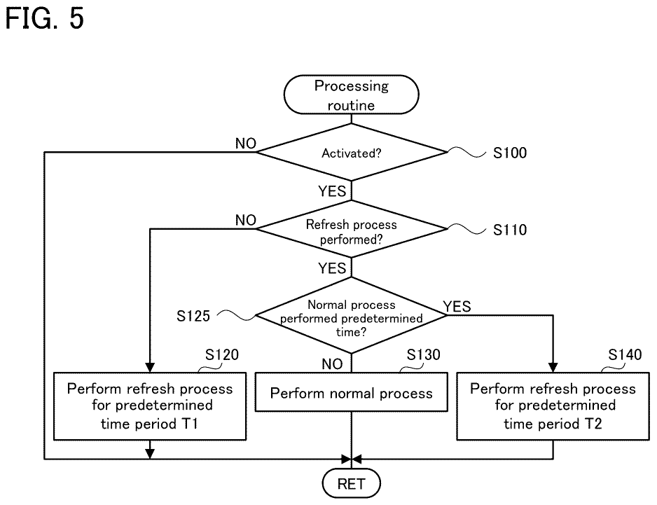

For example, in the embodiment described above, the CPU 97 executes the processing routine of FIG. 3. However, alternatively, the CPU 97 may execute a processing routine of FIG. 5. The processing routine of FIG. 5 is identical to the processing routine of FIG. 3, except that processes of steps S125 and S140 are added. Accordingly, for processes in the processing routine of FIG. 5 that are identical to those in the processing routine of FIG. 3, the same step numbers are assigned, and detailed descriptions thereof are omitted.

In the processing routine of FIG. 5, when the CPU 97 determines in step S110 that the refresh process has already been performed in the current use of the gas sensor 100, the CPU 97 determines whether or not a normal process has been continuously performed for a predetermined time ΔT (that is, whether or not a predetermined time ΔT has elapsed since the previous refresh process) (step S125). Here, in this routine, the expression “continuously performed the normal process for the predetermined time ΔT” means that the normal process (step S130) has been repeatedly performed for the predetermined time ΔT without performing the refresh process. As the predetermined time ΔT, a period of several seconds to several minutes is used. When the CPU 97 determines that the normal process has not been continuously performed for the predetermined time ΔT, the CPU 97 performs the normal process (step S130) and terminates this routine.

When the CPU 97 determines in step S125 that the normal process has been continuously performed for the predetermined time ΔT, the CPU 97 performs a refresh process for a predetermined time T2 (step S140) and terminates this routine. Here, the predetermined time T2 is defined as a time equal to or shorter than the predetermined time T1, and, for example, a period of several milliseconds to several seconds is used. Accordingly, the refresh process can be periodically performed (at intervals of the predetermined time ΔT). As a result, it is possible to ensure the frequency of the refresh process and to suppress a decrease in measurement accuracy of water concentration and carbon dioxide concentration in the measurement gas.

In the processing routine of FIG. 5, the CPU 97 performs the refresh process for the predetermined time T1 when the solid electrolyte of the sensor element 101 is activated (step S120), and thereafter, performs the refresh process for the predetermined time T2 each time the normal process has been continuously performed for the predetermined time ΔT (step S140). However, the CPU 97 may not necessarily perform the refresh process for the predetermined time T1.

In the embodiment described above, the CPU 97 determines whether or not the solid electrolyte of the sensor element 101 is activated by determining whether or not the resistance value of the heater 72 is equal to or less than a predetermined resistance value. However, the present invention is not limited thereto. For example, the CPU 97 may determine whether or not the solid electrolyte is activated by determining whether or not a performance time of the heater control process is equal to or longer than a predetermined time.

In the embodiment described above, the CPU 97 controls the main pump cell 21, as the first refresh process, by feedback-controlling the voltage Vp0 of the variable power supply 24 so that the voltage V0 becomes a target value V0r* whose absolute value is smaller than that of the target value V0*. However, the present invention is not limited thereto. For example, the CPU 97 may control the main pump cell 21, as the first refresh process, by feedback-controlling the voltage Vp0 of the variable power supply 24 so that a pump current Ip0 becomes a target value Ip0r*. The target value Ip0r* is a value in a direction in which oxygen is pumped into the first internal cavity 20, and is defined as a value having a polarity opposite to that in performance of the main pump control process.

In the embodiment described above, the CPU 97 controls the first measurement pump cell 50, as the second refresh process, by feedback-controlling the voltage Vp1 of the variable power supply 52 so that the voltage V1 becomes a target value V1r* whose absolute value is smaller than that of the target value V1*; however, the present invention is not limited thereto. For example, the CPU 97 may control the first measurement pump cell 50, as the second refresh process, by feedback-controlling the voltage Vp1 of the variable power supply 52 so that a pump current Ip1 becomes a target value Ip1r*. The target value Ip1r* is defined as a value whose absolute value is greater than that of the pump current Ip1 normally flowing during performance of the first measurement pump control process.

In the embodiment described above, the CPU 97 controls the second measurement pump cell 41, as the third refresh process, by feedback-controlling the voltage Vp2 of the variable power supply 46 so that the voltage V2 becomes a target value V2r* whose absolute value is smaller than that of the target value V2*. However, the present invention is not limited thereto. For example, the CPU 97 may control the second measurement pump cell 41, as the third refresh process, by feedback-controlling the voltage Vp2 of the variable power supply 46 so that a pump current Ip2 becomes a target value Ip2r*. The target value Ip2r* is defined as a value whose absolute value is greater than that of the pump current Ip2 normally flowing during performance of the second measurement pump control process.

In the embodiment described above, the CPU 97 performs the first, second, and third refresh processes as the refresh process. However, as the refresh process, only a part of the first, second, and third refresh processes may be performed. The inventors have found through experiments and analyses that, during use or stop of the gas sensor 100, among the inner pump electrode 22, the first measurement electrode 51, and the second measurement electrode 44, the inner pump electrode 22 in particular tends to adsorb a gas, which causes a decrease in its reducing capability. Therefore, when only a part of the first, second, and third refresh processes is performed as the refresh process, it is preferable to perform at least the first refresh process.

In the embodiment described above, the CPU 97 measures a water concentration and a carbon dioxide concentration in the measurement gas by performing the water concentration measurement process and the carbon dioxide concentration measurement process. However, the control device 95 may measure only one of the water concentration and the carbon dioxide concentration in the measurement gas by performing only one of the water concentration measurement process and the carbon dioxide concentration measurement process.

In the embodiment described above, the outer pump electrode 23 serves in a dual role as a first outer electrode paired with the inner pump electrode 22 in the main pump cell 21, as a second outer electrode paired with the first measurement electrode 51 in the first measurement pump cell 50, and as a third outer electrode paired with the second measurement electrode 44 in the second measurement pump cell 41. That is, the first to third outer electrodes are configured as a common outer pump electrode 23. However, the present invention is not limited thereto. For example, two of the first to third outer electrodes may be configured as the common outer pump electrode 23, and the remaining one may be provided as an electrode independent of the outer pump electrode 23 on an outer surface of the element body 102 to be in contact with the measurement gas. Alternatively, the first to third outer electrodes may each be provided as independent electrodes on the outer surface of the element body 102 to be in contact with the measurement gas.

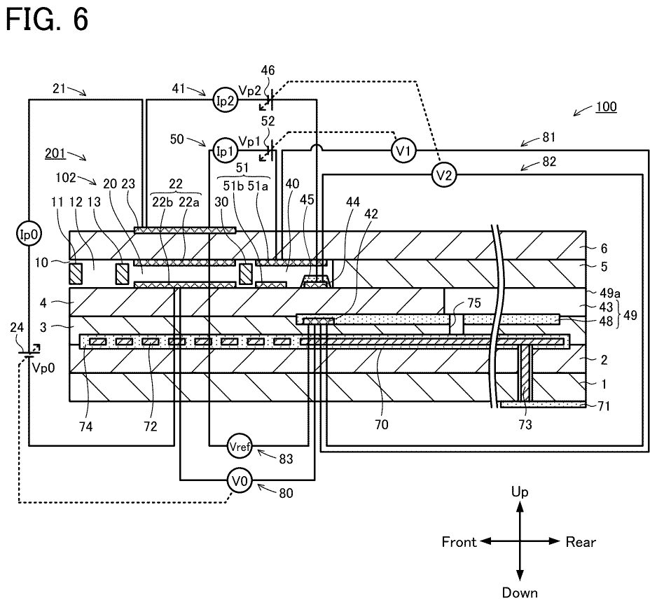

In the embodiment described above, the sensor element 101 of the gas sensor 100 includes the first internal cavity 20, the second internal cavity 40, and the third internal cavity 61. However, the present invention is not limited thereto. For example, as shown in a modified example of a sensor element 201 in FIG. 6, the sensor element may not include the third internal cavity 61. In the sensor element 201 of the modified example shown in FIG. 6, a gas inlet 10, a first diffusion rate-limiting section 11, a buffer space 12, a second diffusion rate-limiting section 13, a first internal cavity 20, a third diffusion rate-limiting section 30, and a second internal cavity 40 are successively and adjacently formed to be in communication with each other in this order between a lower surface of the second solid electrolyte layer 6 and an upper surface of the first solid electrolyte layer 4. The second measurement electrode 44 is disposed on the upper surface of the first solid electrolyte layer 4 within the second internal cavity 40. The second measurement electrode 44 is covered with a fourth diffusion rate-limiting section 45. The fourth diffusion rate-limiting section 45 is a film composed of a ceramic porous body such as alumina (Al₂O₃). The fourth diffusion rate-limiting section 45, similar to the fourth diffusion rate-limiting section 60 in the above-described embodiment, imparts a predetermined diffusion resistance to the measurement gas in the second internal cavity 40 and guides the gas to the second measurement electrode 44. The fourth diffusion rate-limiting section 45 also functions as a protective film for the second measurement electrode 44. A ceiling electrode portion 51a of the first measurement electrode 51 is formed to extend to a position directly above the second measurement electrode 44. Even with the sensor element 201 having such a configuration, it is possible, similarly to the above-described embodiment, to measure a carbon dioxide concentration based on a pump current Ip2 flowing through the second measurement pump cell 41. In the sensor element 201 of FIG. 6, a region around the second measurement electrode 44 functions as a third chamber; that is, the region around the second measurement electrode 44 serves the same role as the third internal cavity 61.

In the embodiment described above, the element body 102 of the sensor element 101 was a laminated body having a plurality of solid electrolyte layers (layers 1 to 6); however, the present invention is not limited thereto. The element body of the sensor element 101 only needs to have at least one solid electrolyte layer having oxygen ion conductivity and to be provided with a measurement gas flow path inside. For example, in FIG. 1, layers 1 to 5 other than the second solid electrolyte layer 6 may be configured as structural layers made of a material other than a solid electrolyte (for example, layers made of alumina). In this case, each electrode included in the sensor element 101 may be disposed on the second solid electrolyte layer 6. For example, the second measurement electrode 44 in FIG. 1 may be disposed on a lower surface of the second solid electrolyte layer 6. In addition, the reference gas introduction space 43 may be provided in the spacer layer 5 instead of in the first solid electrolyte layer 4, the reference gas introduction layer 48 may be provided between the second solid electrolyte layer 6 and the spacer layer 5 instead of between the first solid electrolyte layer 4 and the third substrate layer 3, and the reference electrode 42 may be provided at a position behind the third internal cavity 61 and on the lower surface of the second solid electrolyte layer 6.

In the embodiment described above, the first diffusion rate-limiting section 11, the second diffusion rate-limiting section 13, and the third diffusion rate-limiting section 30 have been provided as two horizontally elongated slits, respectively. However, the present invention is not limited thereto. For example, at least one of the first diffusion rate-limiting section 11, the second diffusion rate-limiting section 13, and the third diffusion rate-limiting section 30 may be provided as a single horizontally elongated slit.

What is claimed is:

1. A gas sensor, comprising a sensor element and a control device, the gas sensor configured to measure at least one of a water concentration and a carbon dioxide concentration in a measurement gas,
wherein the sensor element includes:
an element body having an oxygen-ion-conductive solid electrolyte layer and a measurement gas flow path provided therein, the measurement gas flow path configured to introduce and flow the measurement gas;
a first pump cell including a first inner electrode disposed in a first chamber in the measurement gas flow path and a first outer electrode disposed on an outer surface of the element body;
a second pump cell including a second inner electrode disposed in a second chamber located downstream of the first chamber in the measurement gas flow path and a second outer electrode disposed on the outer surface of the element body; and
a third pump cell including a third inner electrode disposed in a third chamber located downstream of the second chamber in the measurement gas flow path and a third outer electrode disposed on the outer surface of the element body;
wherein the control device is configured to perform:
a first pump cell control process for controlling the first pump cell to pump out oxygen from around the first inner electrode to around the first outer electrode, thereby reducing water and carbon dioxide in the measurement gas in the first chamber;
a second pump cell control process for controlling the second pump cell to pump in oxygen from around the second outer electrode to around the second inner electrode, thereby oxidizing hydrogen generated by reduction of water in the first chamber in the second chamber;
a third pump cell control process for controlling the third pump cell to pump in oxygen from around the third outer electrode to around the third inner electrode, thereby oxidizing carbon monoxide generated by reduction of carbon dioxide in the first chamber in the third chamber; and
at least one of a water concentration measurement process for measuring a water concentration in the measurement gas based on a second pump current flowing through the second pump cell in the second pump cell control process, and a carbon dioxide concentration measurement process for measuring a carbon dioxide concentration in the measurement gas based on a third pump current flowing through the third pump cell in the third pump cell control process;
wherein, when a predetermined condition is satisfied, the control device is configured to perform, as a refresh process, at least one of:
a first refresh process for controlling the first pump cell to pump in oxygen from around the first outer electrode to around the first inner electrode;
a second refresh process for controlling the second pump cell to pump in more oxygen from around the second outer electrode to around the second inner electrode than in the second pump cell control process; and
a third refresh process for controlling the third pump cell to pump in more oxygen from around the third outer electrode to around the third inner electrode than in the third pump cell control process.

2. The gas sensor according to claim 1,
wherein the control device is configured to perform at least the first refresh process as the refresh process.

3. The gas sensor according to claim 1,
wherein the predetermined condition includes a condition in which the solid electrolyte layer is activated.

4. The gas sensor according to claim 1,
wherein the predetermined condition includes a condition in which the first, second, and third pump cell control processes have been continuously performed for a predetermined period of time.

5. The gas sensor according to claim 1,
wherein at least two of the first, second, and third outer electrodes are implemented as a single common electrode.
